(19) United States
(12) Patent Application Publication
(10) Pub. No.: US 2026/0076884 A1
(43) Pub. Date: Mar. 19, 2026

(54) DRY SHAMPOO AND RELATED METHODS

(71) Applicant: PLZ Corp., Downers Grove, IL (US)

(72) Inventors: Ian Joshua FISHMAN, Riverside, CA (US); Jorge Alfredo POLANCO, Riverside, CA (US); Alfonso Cecilio MORENO, Riverside, CA (US)

(21) Appl. No.: 19/326,211

(22) Filed: Sep. 11, 2025

Related U.S. Application Data
(60) Provisional application No. 63/694,373, filed on Sep. 13, 2024.

(51) Int. Cl. A61K 8/33; A61K 8/04; A61K 8/25; A61K 8/34; A61K 8/37; A61K 8/73; A61Q 5/02

(52) U.S. Cl. CPC A61K 8/33; A61K 8/046; A61K 8/25; A61K 8/34; A61K 8/37; A61K 8/732; A61Q 5/02; A61K 2800/30

(57) ABSTRACT

This disclosure relates to dry shampoo compositions. In particular, the present disclosure pertains to dry shampoo compositions that are environmentally friendly yet effective. The dry shampoo compositions provided herein may function, in part, by absorbing excess oil and sebum from the scalp and hair, thereby reducing the appearance of greasiness and extending the time between conventional washes.

### CROSS-REFERENCE TO RELATED APPLICATIONS

The present application claims priority to U.S. Application No. 63/694,373 filed Sep. 13, 2024, the contents of which are incorporated herein.

### BACKGROUND

Dry shampoos have gained widespread popularity as a convenient alternative to traditional shampoos, allowing users to refresh their hair without needing water. Originally developed as a time-saving alternative to traditional shampoo, dry shampoo absorbs excess oil and adds volume to hair without water. While dry shampoo formulations have evolved significantly with recent advancements focusing on enhancing formulation effectiveness, there remains a need, driven by both consumer demand and regulatory changes, to provide products that also meet environmental standards.

Some dry shampoo formulations feature natural and organic ingredients, which cater to the rising consumer preference for clean beauty products. For example, some dry shampoo formulations incorporate plant-based ingredients and avoid harsh chemicals like sulfates and parabens. There is also an ongoing shift towards water-less formulations, which are more sustainable since they reduce water use during production and require less packaging. Additionally, the trend of offering customizable formulations is gaining traction, allowing users to select dry shampoos tailored to their specific hair type and needs.

In addition to ingredient reformulation, there is a broader trend towards sustainability across the personal care industry. Consumers are demanding products that are not only effective but also environmentally responsible. As a result, dry shampoos are often free from sulfates, parabens, and other harmful chemicals, with an increased focus on using natural and biodegradable ingredients. These changes are aligned with the clean beauty movement, which prioritizes transparency and safety in product formulations.

Dry shampoos that include high concentrations of ingredients like alcohols (e.g., ethanol) and hydrofluorocarbons (e.g., HFC-152a) may be efficient, but contribute to a higher environmental impact. With recent changes, there's a shift toward using more eco-friendly alternatives that still deliver the desired results.

One critical aspect influencing modern formulations is the Maximum Incremental Reactivity (MIR) metric. MIR quantifies the potential of volatile organic compounds (VOCs) in a product to contribute to ozone formation, a key factor in air quality management. As environmental concerns grow, regulatory agencies such as the California Air Resources Board (CARB) have tightened limits on VOCs in consumer products, including dry shampoos. For example, California's new VOC limits mandate a maximum of 55% VOCs in dry shampoos, effective from 2023, with even stricter limits expected by 2029.

### SUMMARY

Dry shampoo compositions are provided that may be used for topical application to hair (e.g., human hair) when the hair is in need to cleaning without the need for water. The dry shampoo compositions provided herein may be formulated as a spray or powder. The spray may be under pressure so as to propel the dry shampoo composition onto the hair upon engagement. The dry shampoo compositions provided herein may function, in part, by absorbing excess oil and sebum from the scalp and hair, thereby reducing the appearance of greasiness and extending the time between conventional washes.

The dry shampoo compositions provided herein deliver considerable convenience and versatility, particularly for individuals with limited access to water or those requiring rapid grooming solutions. The dry shampoo compositions provided herein may be subject to repeated use but prevents buildup on the scalp or hair shaft thereby avoiding irritation or obstruction of hair follicles. The dry shampoo compositions provided herein avoid noticeable residue, especially on darker hair. Finally, the dry shampoo compositions provided herein meet or exceed regulatory and safety requirements relevant to dry shampoos.

According to one aspect, the present disclosure provides various dry shampoo compositions that may optionally include one or more of each of the following components: propellants, hair fixatives, emollients, oil-absorbing agents, solvents, preservatives, neutralizing agents, chelating agents, and fragrances.

According to another aspect, the present disclosure provides methods of refreshing or cleansing hair without water. The methods include the steps of providing a dry shampoo composition as described herein and dispensing the dry shampoo composition to the scalp or hair of a subject in need thereof to reduce oiliness and refresh the hair without rinsing.

In yet another aspect, the present disclosure provides a dry shampoo product including a container, a dry shampoo composition according to the present disclosure located in an interior product storage region of the container, and a spraying device including a stem orifice, an actuator orifice, and optionally a vapor tap orifice.

Additional embodiments, features, and advantages of the disclosure will be apparent from the following detailed description and through practice of the disclosure. The compounds and methods of the present disclosure can be described as embodiments in any of the following enumerated clauses. It will be understood that any of the embodiments described herein can be used in connection with any other embodiments described herein to the extent that the embodiments do not contradict one another.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Definitions

Unless otherwise defined herein, scientific and technical terms used in this application shall have the meanings that are commonly understood by those of ordinary skill in the art. Generally, nomenclature used in connection with, and techniques of chemistry described herein are those well-known and commonly used in the art.

The methods and techniques of the present disclosure are generally performed, unless otherwise indicated, according to conventional methods well known in the art and as described in various general and more specific references that are cited and discussed throughout this specification.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

As used herein and in the appended clauses, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

As used herein, the term “halo” or “halogen” means fluoro, chloro, bromo, or iodo, each of which may be used interchangeably with the terms F, Cl, Br or I, respectively.

Chlorofluorocarbons (CFCs) and hydrochlorofluorocarbons (HCFCs) refer to fully or partly halogenated hydrocarbons that contain carbon (C), hydrogen (H), chlorine (CI), and fluorine (F), produced as volatile derivatives of methane, ethane, and propane. Non-limiting examples of CFCs and HCFCs include trichlorofluoromethane (CFC-11), dichlorodifluoromethane (CFC-12), 1,1,2-trichloro-1,2,2-trifluoroethane (CFC-113), 1,2-dichloro-1,1,2,2-tetrafluoroethane (CFC-114), chloropentafluoroethane (CFC-115), chlorodifluoromethane (HCFC-22), 1,1,1-trifluoro-2,2-dichloroethane (HCFC-123), 1,1-dichloro-1-fluoroethane (HCFC-141b), 1-chloro-1,1-difluoroethane (HCFC-142b), and 2-chloro-1, 1,1,2-tetrafluoroethane (HCFC-124),

Hydrofluorocarbons (HFCs) refer to synthetic organic compounds that contain fluorine and hydrogen atoms, and are the most common type of organofluorine compounds. Non-limiting examples of HFCs include trifluoromethane (HFC-23), 1,1,2,2-tetrafluoroethane (HFC-134), 1,1,1,2-tetrafluoroethane (HFC-134a), pentafluoroethane (HFC-125), 1,1,1-trifluoroethane (HFC-143a), 1,1-difluoroethane (HFC-152a), ethoxy-nonafluorobutane (HFE 7200), trans-1,3,3,3-tetrafluoropropene (HFO-1234ze), and perfluorocarbons.

“Global Warming Potential (GWP)” means the radiative forcing impact of one mass-based unit of a given greenhouse gas relative to an equivalent unit of carbon dioxide over a given period of time (e.g., one hundred years). For example, HFC-152a has a GWP of 124, meaning it is 124 times more potent than carbon dioxide over a 100-year period.

The terms “hair-fixative polymer” or “hair fixative” are given their ordinary meaning in the art and generally refer to a film-forming polymer which is soluble in selected solvent(s) (e.g., present in the dry shampoo composition) and is capable of forming a film and/or holding the hair of a user in place after evaporation of the selected solvent(s). Non-limiting examples of hair fixative polymers include octylacrylamide/acrylates/butylaminoethyl/methacrylate copolymer, PVP, VP/VA copolymer, PVP/VA copolymer, acrylates copolymer, polyurethane-14/AMP-acrylates copolymer, esters of PVM/MA copolymer, and combinations thereof. Other suitable hair fixatives will be known to those of ordinary skill in the art.

“Hair Styling Product” means a consumer product that is designed or labeled for application to wet, damp or dry hair to aid in defining, shaping, lifting, styling and/or sculpting of the hair. “Hair Styling Product” includes, but is not limited to, hair balm, clay, cream, creme, curl straightener, gel, liquid, lotion, paste, pomade, putty, root lifter, serum, spray gel, stick, temporary hair straightener, wax, spray products that aid in styling but do not provide finishing of a hair style, and leave-in volumizers, detanglers and/or conditioners that make styling claims.

“Hair Styling Product” does not include “Dry Shampoo,” “Thermal Protectant,” “Hair Mousse,” “Hair Shine,” “Hair Finishing Spray,” or shampoos or conditioners that are rinsed from the hair prior to styling.

“Head space” refers to the safety margin to allow for expansion in a pressurized container.

“Finish” or “finishing” means the maintaining and/or holding of a hairstyle once all styling is complete.

“Styling” means forming, sculpting, or manipulating the hair to alter the hair's shape.

“Maximum Incremental Reactivity (MIR)” refers to the quantifiable metric used to evaluate the ozone-forming potential of a specific volatile organic compound (VOC) when introduced into a controlled mixture of reactive organic gases (e.g., a base ROG mixture). The MIR value is defined as the maximum increase in the mass of ozone produced, expressed in grams of ozone (g O₃), per gram of the VOC added (g VOC). The MIR metric is utilized primarily to assess and compare the relative contribution of different VOCs to photochemical smog formation. The calculation of MIR involves the addition of a defined quantity of the VOC to a Base ROG Mixture, under conditions that replicate atmospheric photochemical reactions, including the presence of sunlight and nitrogen oxides (NOx). The resulting change in ozone concentration, observed within a specified period, is then divided by the mass of the VOC added to determine the MIR value pursuant to the following formula:

$$MIR = Total Incremental {O}_{3} ( g ) Formed ( g ) / VOC Added ( g )$$

MIR values may be determined based on established scientific models, such as those developed or published by the California Air Resources Board (CARB), or other recognized regulatory or scientific authorities. For purposes of the present disclosure, MIR values are used to assess, control, or limit the aggregate ozone-forming potential of compositions containing one or more VOCs.

“Propellant” means a liquefied or compressed gas that is used in whole or in part, such as a cosolvent, to expel a liquid or any other material from the same self-pressurized container or from a separate container.

“Type A Propellant” means a compressed gas such as CO2, N2, N2O, or compressed air, which is used as a propellant, and is either incorporated with the product or contained in a separate chamber within the product's packaging.

“Type B Propellant” means any halocarbon which is used as a propellant including chlorofluorocarbons (CFCs), hydrochlorofluorocarbons (HCFCs), and hydrofluorocarbons (HFCs).

“Type C Propellant” means any propellant which is not a Type A or Type B propellant, including propane, isobutane, n-butane, and dimethyl ether (also known as dimethyl oxide).

“Volatile Organic Compound (VOC)” means any compound containing at least one atom of carbon, excluding carbon monoxide, carbon dioxide, carbonic acid, metallic carbides or carbonates, and ammonium carbonate, and excluding the following:

“VOC Content” means the total weight of VOC in a product expressed as a percentage of the product weight (exclusive of the container or packaging).

The phrases “wt %,” “percent by weight,” “by weight percent” are used interchangeably and refer to the percent of a component based on the total weight of the composition.

### Overview

The present disclosure provides various dry shampoo compositions suitable for a variety of hair care and styling applications. According to one embodiment, the dry shampoo compositions may provide multiple cosmetic and functional benefits to the hair following application. The compositions may be applied to help refresh the appearance and feel of hair by absorbing excess oil and sebum at the scalp and along the hair shaft, thereby extending the time between traditional washes. In addition to oil control, the dry shampoo compositions may improve the visual appearance of the hair by enhancing volume, texture, and body while reducing the appearance of greasiness or flatness. The compositions may include conditioning agents, anti-static compounds, or protective additives such as UV filters to support the overall health and manageability of the hair. Unlike traditional dry shampoos that may leave behind a heavy or chalky residue, the dry shampoo compositions provided herein are formulated to leave a clean, lightweight finish without visible buildup or flaking. The compositions exhibit rapid drying following application and leave the hair soft to the touch with a refreshed, non-tacky feel.

The dry shampoo compositions provided herein exhibit a maximum incremental reactivity (MIR) of 0.75, 0.74, 0.73, 0.72, 0.71, 0.70, or less. According to one embodiment, the dry shampoo compositions may have an MIR of less than about 0.70, less than about 0.65, or less than about 0.60. In some embodiments, the compositions may have an MIR from about 0.40 to about 0.79, about 0.50 to about 0.78, or about 0.50 to about 0.75.

In certain embodiments, the dry shampoo compositions include only components with a Global Warming Potential (GWP) of less than 10. According to one embodiment, the compositions include only components with a GWP of less than about 10, less than about 5, or less than about 2. In certain embodiments, the compositions include only components with a GWP of 0. In certain embodiments, the dry shampoo composition has a GWP equal to or less than 150.

In some embodiments, the dry shampoo compositions are substantially free or completely free of 1,1-difluoroethane (HFC-152a). In some embodiments, the compositions do not include one or more of a Type A, Type B, or Type C propellant. In some embodiments, the compositions may include minimal amounts of one or more of a Type A, Type B, or Type C propellant such that the MIR is maintained at or below 0.74. In some embodiments, the dry shampoo compositions include primarily non-VOC propellants to reduce ozone formation potential while maintaining a MIR of less than 0.75.

Various dry shampoo compositions are provided and may be incorporated in aerosol or non-aerosol delivery formats for use in both consumer and professional hair care products. The dry shampoo compositions of the present disclosure may include water-based or water-free formulations optimized for performance, environmental safety, and cosmetic elegance.

### A. Propellant

The dry shampoo compositions provided herein include one or more propellants. Non-limiting examples of propellants include liquefied gas propellants such as alkanes (e.g., ethane, propane, n-butane, isobutane, n-pentane, iso-pentane, blends of hydrocarbons (e.g., C₃-C₇ hydrocarbons), liquefied petroleum gas), and ethers (e.g., dimethyl ether), and compressed gas propellants such as carbon dioxide, nitrous oxide, nitrogen. In some embodiments, the propellant is a liquefied gas propellant. For example, the liquefied gas propellant may be an alkane, an ether, or a combination thereof.

In some embodiments, the propellant includes at least one of an alkane (e.g., a hydrocarbon) or an ether. For example, the propellant may include an alkane blend of two or more (i.e., 2, 3, or 4) alkanes. In some embodiments, the propellant includes an alkane blend, the alkane blend including a first alkane and a second alkane that is different than the first alkane. For example, the propellant may include a first alkane (e.g., propane) and a second alkane (e.g., butane, such as n-butane, iso-butane). In some embodiments, the propellant includes propane and butane. In some embodiments, the propellant includes a hydrocarbon blend of alkanes (e.g., a mixture of C3-C7 hydrocarbons). In some embodiments, the propellant includes an ether (e.g., dimethyl ether). In some embodiments, the propellant may include A-17 (n-butane) available from Aeropres Corp., A-31 (iso-butane) available from Aeropres Corp., A-108 (propane) available from Aeropres Corp., AB-70 (50% propane/50% n-butane alkane blend) available from Aeropres Corp., or dimethyl ether available from Diversified CPC International, Inc.

In some embodiments, the dry shampoo compositions include various weights of propellant as set forth in the ranges in the exemplary embodiments set forth herein. In some embodiments, the dry shampoo compositions include about 10.0% to about 90.0% by weight of the propellant. For example, the dry shampoo compositions may include greater than about 30.0%, about 45.0%, about 40.0%, about 50.0%, about 55.0%, about 60.0%, about 65.0%, about 70.0%, about 75.0%, about 80.0%, or about 85.0% propellant. In other embodiments, the dry shampoo compositions may include less than about 90.0%, about 85.0%, about 80.0%, about 75.0%, about 70.0%, about 65.0%, about 60.0%, about 55.0%, about 50.0%, about 45.0%, about 40.0%, about 35.0%, or about 30.0% propellant. In some embodiments, the dry shampoo compositions may include from about 30.0% to about 90.0%, about 35.0% to about 85.0%, about 35.0% to about 80.0%, about 40.0% to about 85.0%, or about 40.0% to about 80.0% by weight of propellant.

In some embodiments, the dry shampoo compositions include about 10.0% to about 90.0% by weight of the propellant. In some water-based embodiments, the dry shampoo compositions include about 40.0%, about 50.0%, about 60.0%, about 65.0%, about 66.0%, about 67.0%, about 68.0%, about 69.0%, about 70.0%, about 75.0%, about 80.0%, about 85.0%, or about 90.0% by weight of propellant. In some embodiments, the dry shampoo compositions include about 50.0% to about 90.0% by weight of the propellant.

### B. Solvents

In some embodiments, the dry shampoo composition includes at least one of a solvent. For example, the dry shampoo composition may include a solvent mixture of two or more (i.e., 2, 3, or 4) solvents. In some embodiments, the dry shampoo composition includes a solvent mixture, the solvent mixture including a first solvent (e.g., an alcohol) and a second solvent (e.g., water) that is different than the first solvent.

In some embodiments, the dry shampoo composition includes about 1.0% to about 40.0% by weight of the solvent. For example, the dry shampoo composition may include less than about 40.0%, less than about 35.0%, less than about 30.0%, less than about 25.0%, less than about 20.0%, less than about 15.0%, or less than about 10.0% by weight of a solvent. In some embodiments, the dry shampoo composition may include less than about 15.0% or less than about 10.0% by weight of a solvent such as alcohol. In some embodiments, the dry shampoo may include from about 5.0% to about 40.0%, from about 5.0% to about 35.0%, from about 5.0% to about 30.0%, from about 5.0% to about 25.0%, from about 5.0% to about 20.0%, or about 5.0% to about 15.0% by weight of solvent. In some embodiments, the dry shampoo composition includes about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 21.0%, about 22.0%, about 23.0%, about 24.0%, about 25.0%, about 26.0%, about 27.0%, about 28.0%, about 29.0%, about 30.0%, about 31.0%, about 32.0%, about 33.0%, about 34.0%, about 35.0% by weight of solvent.

In some embodiments, the solvent is an alcohol. Non-limiting examples of alcohols include ethanol, propanol, and butanol. In certain embodiments, the alcohol is propanol (e.g., isopropanol). In some embodiments, the alcohol may include 99% isopropanol (isopropyl alcohol-IPA) available from Sigma-Aldrich. In some embodiments, the solvent is an alkane (e.g., isohexadecane, and n-dodecane).

In some embodiments, the dry shampoo composition includes about 5% to about 20% by weight of the alcohol. For example, the dry shampoo composition may include less than about 20%, less than about 15%, less than about 12.5%, about 5% to about 15% or about 7.5% to about 12.5% by weight of alcohol. In some embodiments, the dry shampoo composition includes about 5%, about 6%, about 7%, about 8%, about 9%, about 10%, about 11%, about 12%, about 13%, about 14%, or about 15% by weight of alcohol.

In some embodiments, the dry shampoo composition includes about 15% to about 35% by weight of water. For example, the dry shampoo composition may include less than about 30%, less than about 25%, or about 20% to about 25% by weight of water. In some embodiments, the dry shampoo composition includes about 15%, about 16%, about 17%, about 18%, about 19%, about 20%, about 21%, about 22%, about 23%, about 24%, about 25%, about 26%, about 27%, about 28%, about 29%, about 30% or more by weight of water.

### C. Preservatives

In some embodiments, the dry shampoo compositions may include at least one preservative (e.g., one, two, three, four, five, or more). Suitable preservatives include, but are not limited to, phenoxyethanol, ethylhexylglycerin, methylparaben, propylparaben, DMDM hydantoin, imidazolidinyl urea, diazolidinyl urea, chlorphenesin, caprylyl glycol, benzyl alcohol, dehydroacetic acid, sodium benzoate, potassium sorbate, sorbic acid, and phenylpropanol. These preservatives may be included individually or in combination in an amount effective to inhibit the growth of microorganisms, such as bacteria, yeasts, and molds, thereby enhancing the microbiological stability and shelf life of the dry shampoo compositions.

In some embodiments, they dry shampoo compositions include at least one preservative as set forth in the ranges in the exemplary embodiments set forth herein. In some embodiments, the dry shampoo compositions include from about 0.01% to about 2.0% by weight of the at least one preservative. For example, the dry shampoo compositions may include less than about 2.0%, about 1.5%, about 1.0%, about 0.75%, about 0.50%, or about 0.1% by weight of the at least one preservative. In some embodiments, the dry shampoo compositions include about 0.01%, about 0.05%, about 0.1%, about 0.3%, about 0.5%, about 0.75%, about 1.0%, about 1.5% or about 2.0% by weight of the at least one preservative. In some embodiments, the dry shampoo compositions include about 0.1% to about 2.0% of the at least one preservative.

### D. Hair Fixatives

In some embodiments, the dry shampoo compositions may include at least one hair fixative (e.g., one, two, three, four, five, or more). The hair fixative may function as a binder or film former in addition to a hair fixative. The hair fixative may include one or more film-forming polymers, copolymers, biopolymers, or resins including, but not limited to, Trimethylsiloxysilicate, PVP Butylated, VP/VA copolymer, Octylacrylamide/Acrylates/Butylaminoethyl Methacrylate Copolymers, Acrylates/Octylacrylamide Copolymer, VA/Crotonated/Vinyl Neodecanoate Copolymer, Polyurethane-14, VP/VA Copolymer, PVP, Polyquaternium-46 Copolymer, Butyl Ester of PVM/MA Copolymer, Acrylates/Hydroxyesters Acrylates Copolymer, Acrylates Copolymer, VP/Vinyl Caprolactam/DMAPA Acrylates Copolymer, Vinyl Caprolactam/VP/Dimethylaminoethylmethacrylate Copolymer, and Methacryloyl Ethyl Betaine/Acrylates Copolymer. These polymers may be used alone or in combination to impart desired properties such as hold, flexibility, humidity resistance, and shine to the finished hair style.

In some embodiments, the dry shampoo compositions may include at least one hair fixative in the ranges in the exemplary embodiments set forth herein. In some embodiments, the dry shampoo compositions include from about 0.001% to about 5.0% by weight of the at least one hair fixative. For example, the dry shampoo compositions may include less than about 5.0%, about 4.0%, about 3.0%, about 2.0%, about 1.0%, about 0.5%, about 0.1%, about 0.01%, or about 0.001% by weight of the at least one hair fixative. In some embodiments, the dry shampoo compositions include about from about 0.1% to about 2.0% by weight of the at least one hair fixative.

### E. Neutralizing Agents

In some embodiments, the dry shampoo compositions may include at least one neutralizing agent. When present, the neutralizing agent may be utilized to adjust or stabilize the pH of the dry shampoo compositions, typically to a level that is compatible with both the hair and scalp (usually in the range of pH about 4.5 to about 9.5 or about 5.5 to about 9.5). The at least one neutralizing agent may react with acidic groups (such as in hair fixative components) to enhance solubility, viscosity control, and film formation, thereby improving the spray's performance, such as hold strength, dry time, and resistance to humidity. Common neutralizing agents include, but are not limited to, amines, aminomethyl propanol, potassium hydroxide, sodium hydroxide, triethanolamine, citric acid, lactic acid (88.0% lactic acid; 12.0% water; food chemical codex (FCC)), and any combination thereof selected based on the target pH, compatibility with other ingredients, and desired performance attributes. In some embodiments, the dry shampoo compositions may include citric acid granular anhydrous (fine) which may additionally function as a chelating agent and preservative. By including at least one neutralizing agent, proper neutralization may be achieved to minimize scalp irritation and ensure the stability and clarity of the dry shampoo compositions.

In some embodiments, the dry shampoo compositions may include at least one neutralizing agent in the ranges in the exemplary embodiments set forth herein. In some embodiments, the dry shampoo compositions include from about 0.0001% to about 15.0% by weight of at least one neutralizing agent. For example, the dry shampoo compositions may include less than about 15.0%, about 12.5%, about 10.0%, about 7.5%, about 5.0%, 2.5%, about 0.50%, about 0.10%, about 0.01%, about 0.001%, or about 0.0001% by weight of at least one neutralizing agent. In some embodiments, the dry shampoo compositions include about 0.0001% to about 0.1% by weight of at least one neutralizing agent.

### F. Emollients

In some embodiments, the dry shampoo compositions may include at least one emollient in the ranges in the exemplary embodiments set forth herein. In some embodiments, the at least one emollient may include DL Panthenol (analog of pantothenic acid (vitamin B5) available from Sigma-Aldric), or Radia 7732 (isopropyl palmitate available from CoastSouthwest) or glycerin (e.g., Refined Glycerine 99.7% MIN USP KSHR available from Wilmar), ZEMEA® (propanediol available from DuPont Tate), C13-C15 alkane (mixture of saturated hydrocarbons), heptyl undecylenate, Ceraphyl 230 (isopropyl isostearate), Cosmosil SBS oil (sea buckthorn seed oil obtained by cold pressing seeds of Hippophaë rhamnoides plant), Omegia (sea buckthorn-derived omega-rich nutraceutical), or any combination thereof.

In some embodiments, the dry shampoo compositions include from about 0.05% to about 6.5% by weight of the at least one emollient. For example, the dry shampoo compositions may include less than about 6.5%, about 6.0%, about 5.0%, about 4.5%, about 4.0%, about 3.5%, about 3.0%, about 2.5%, about 2.0%, about 1.5%, about 1.0%, about 0.5%, or about 0.10%, or about 0.01% by weight of the at least one emollient. In some embodiments, the dry shampoo compositions include about 0.01% to about 6.0% or from about 0.05% to about 5.0% by weight of the at least one emollient.

### G. Chelating Agents

In some embodiments, the dry shampoo compositions may include at least one chelating agent. The at least one chelating agent may aid in surfactant performance. At least one suitable chelating agent includes tetrasodium glutamate diacetate or Pollout® Detox available from Chemyunion which includes from about 35.0 wt % to about 63.9 wt % sodium polyitaconate, about 35.0 wt % to about 61.0 wt % sodium citrate and about 0.10 wt % to about 5.0 wt % Sapindus mukorossi fruit extract. In some embodiments, the dry shampoo compositions include from about 0.01% to about 10.0% by weight of the at least one chelating agent. In some embodiments, the dry shampoo compositions include up to about 5.0% by weight of the at least one chelating agent. In some embodiments, the dry shampoo compositions include from about 0.05% to about 5.0% by weight of the at least one chelating agent.

### H. Fragrances

In some embodiments, the dry shampoo composition may include one or more fragrances to mask any unpleasant odors and enhance the overall sensory experience for the user. Suitable fragrances include, but are not limited to, terpinolene, α-terpineol, linalool, geraniol, citronellol, limonene, eucalyptol, β-pinene, myrcene, and p-cymene. These fragrance compounds may be selected based on their compatibility with hair care formulations and their ability to impart fresh, herbaceous, citrus, or floral olfactory notes like those of terpinolene. Each of the listed fragrance components may be used individually or in combination to enhance the sensorial profile of the composition, while maintaining stability and performance.

In some embodiments, the dry shampoo compositions may include at least one fragrance in the ranges in the exemplary embodiments set forth herein. In some embodiments, the dry shampoo compositions include from about 0.01% to about 3.0% by weight of the at least one fragrance. For example, the dry shampoo compositions may include less than about 3.0%, about 2.0%, about 1.5%, about 1.0%, about 0.75%, about 0.50%, or about 0.1% by weight of the at least one fragrance. In some embodiments, the dry shampoo composition includes about 0.01%, about 0.05%, about 0.1%, about 0.5%, about 0.75%, about 1.0%, about 1.5% or about 2.0% by weight of the at least one fragrance.

### I. Oil-Absorbing Components

In some embodiments, the dry shampoo compositions may include at least one oil-absorbing component. In some embodiments, the oil-absorbing component may be a powder. In certain embodiments, the oil-absorbing component includes a starch (e.g., potato starch, rice starch, tapioca starch, wheat starch, or cornstarch). The oil-absorbing component may include at least one type of starch. For example, it may include a mixture of two or more starches (i.e., two, three, or four starches). In some embodiments, the oil-absorbing component includes a starch mixture that includes a first starch and a second starch that is different from the first starch. For instance, the oil-absorbing component may include a first starch (e.g., rice starch) and a second starch (e.g., potato starch). In other embodiments, the oil-absorbing component includes charcoal, alumina, cellulose (e.g., hydroxyethylcellulose), carbonate, hectorite, clay, silica, capsoil, hydrolyzed corn starch, or a combination thereof.

Non-limiting examples of starches include cornstarch, potato starch, tapioca starch, rice starch, wheat starch, and cassava starch. Starches may be characterized by two distinct granule types: A-type starch (AS), with granules larger than 10.0 μm in diameter, and B-type starch (BS), with granules measuring no more than 10.0 μm in diameter. In some embodiments, the starch may also function as an oil absorber. The starch described herein may be modified or unmodified. A modified starch is one that has been derivatized or physically or chemically altered by processes familiar to those skilled in the art, such as esterification, etherification, oxidation, acid hydrolysis, crosslinking, or enzyme conversion. Non-limiting examples of modified starches include aluminum starch octenylsuccinate, aluminum starch octenylsuccinate with acrylates copolymer and magnesium carbonate, sodium starch octenylsuccinate, calcium starch octenylsuccinate (e.g., modified cornstarch), distarch phosphate, hydroxyethyl starch phosphate, hydroxypropyl starch phosphate, sodium carboxymethyl starch, dimethylimidazolidinone starch (e.g., modified rice starch), starch with cetrimonium chloride (e.g., modified rice starch), and sodium starch glycolate. It should be understood, however, that in some embodiments, starch may be replaced by, or used in conjunction with, another oil-absorbing component such as cellulose, maltodextrin, magnesium carbonate, chalk (e.g., calcium carbonate), talc, or fuller's earth. In some embodiments, the component may include DSA 79092 (Oryza sativa (rice) starch with cetrimonium chloride) available from Agrana USA, rice starch NS (dimethylimidazolidinone rice starch) available from Agrana USA, potato starch B available from Roquette, NATRASORB® HFB (aluminum starch octenylsuccinate, acrylates copolymer, magnesium carbonate) available from Nouryon, or FARMAL™ AF 1100 (calcium starch octenylsuccinate), available from Ingredion.

In some embodiments, the oil-absorbing component includes silica (e.g., silica powder). In certain embodiments, the silica includes a first silica and a second silica that is different from the first. Silica (e.g., as a suspending agent, cleaning agent, or oil-absorbing agent) may include Aerosil 200 F available from Evonik (hydrophilic fumed silica with a specific surface area of from about 175 m²/g to about 200 m²/g), Cabo-O-Sil (fumed silica; synthetic amorphous, pyrogenic silica) available from Cabot, Spherilex 10PC available from Evonik (hydrated silica; precipitated silicon dioxide), SPHERON P-1500 available from Presperse (amorphous silica), LOR Bentone 38V CG (modified hectorite clay including magnesium silicate with lithium and sodium ions), or Solesphere H-53 available from Resifa (amorphous silica).

In some embodiments, the dry shampoo composition includes about 0.01% to about 5.0% by weight of silica. For example, the composition may include greater than about 0.01%, greater than about 0.05%, or greater than about 0.1% and less than about 5.0%, less than about 3.0%, about 0.01% to about 5.0%, about 0.01% to about 4.0%, about 0.01% to about 3.0%, about 0.01% to about 2.0%, about 0.01% to about 1.0%, about 0.05% to about 5.0%, about 0.05% to about 4.0%, about 0.05% to about 3.0%, about 0.05% to about 2.0%, about 0.05% to about 1.0%, about 0.1% to about 5.0%, about 0.1% to about 4.0%, about 0.1% to about 3.0%, about 0.1% to about 2.0%, about 0.1% to about 1.0%, about 0.5% to about 5.0%, about 0.5% to about 4.0%, about 0.5% to about 3.0%, about 0.5% to about 2.0%, or about 0.5% to about 1.0% by weight of silica.

In some embodiments, the dry shampoo composition includes about 1.0% to about 20.0% by weight of the oil-absorbing component. For example, the dry shampoo composition may include less than about 20.0%, less than about 15.0%, less than about 12.5%, less than about 10.0%, less than about 9.0%, less than about 8.0%, less than about 7.0%, or less than about 6.0% by weight of oil-absorbing component. In some embodiments, the dry shampoo composition includes about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, or about 15.0% by weight of oil-absorbing component. In some embodiments, the dry shampoo composition includes from about 2.0% to about 6.0% by weight of oil-absorbing component.

In some embodiments, an oil-absorbing component (e.g., a starch and/or silica) and optionally an additive may be combined to form a concentrate. In other embodiments, the concentrate includes an oil-absorbing component (e.g., a starch and/or silica), and optionally silk, zeolite, or a combination thereof. The concentrate may include about 90.0% to about 100.0% by weight of an oil-absorbing component. Alternatively, the concentrate may include about 60.0% to about 100.0%, about 70.0% to about 100.0%, or about 80.0% to about 100.0% by weight of oil-absorbing component. For example, the concentrate may include about 90.0% to about 100.0% by weight of an oil-absorbing component (e.g., approximately 95.0% by weight) and up to about 10.0% by weight of an additive (e.g., approximately 5.0% by weight).

### J. Suncreeen

In some embodiments, the dry shampoo compositions may include at least one sunscreen component so as to render a sun protection factor (SPF) infused dry shampoo composition. The sunscreen component may be useful to prevent sunburn on the scalp without leaving greasy or heavy film on the hair and scalp.

Non-limiting examples of sunscreen components include Avobenzone USP (butyl methoxydibenzoylmethane), Parsol HMS (3,3,5-Trimethylcyclohexyl salicylate), Parsol EHS (ethylhexyl salicylate), octocrylene, zinc dioxide, titanium dioxide, and Zinclear XP (zinc oxide).

The amount of sunscreen component present in the dry shampoo compositions may vary based on location/country of manufacture and use. In some embodiments, the dry shampoo composition includes less than about 25.0%, 20.0%, 15.0%, 10.0%, 5.0%, 2.0%, 1.0% by weight of one or more sunscreen components. In some embodiments, the dry shampoo composition includes up to about 25.0%, 20.0%, 15.0%, 10.0%, 5.0%, 2.0% or 1.0% by weight of one or more sunscreen components.

### K. Additives and Other Components

In some embodiments, the dry shampoo compositions may include at least one additive in the ranges in the exemplary embodiments set forth herein.

In some embodiments, the dry shampoo compositions may include at least one emulsifier. In certain embodiments, the hair styling composition may include one or more emulsifiers to facilitate the formation and stabilization of emulsions that include immiscible phases, such as oil-in-water or water-in-oil systems. Emulsifiers serve to reduce interfacial tension between the aqueous and lipid components, thereby ensuring homogeneous distribution of functional ingredients and enhancing formulation stability over time. Suitable emulsifiers may include PEG-40 hydrogenated castor oil, a nonionic surfactant derived from castor oil that has been ethoxylated and hydrogenated to improve its solubility, mildness, and emulsifying capacity. PEG-40 hydrogenated castor oil functions effectively across a broad pH range and is compatible with a wide variety of oils, fragrances, and conditioning agents. It is particularly useful for solubilizing fragrance oils and lipophilic active ingredients into aqueous systems, providing clarity and stability in transparent formulations. Another suitable emulsifier is C12-C15 Pareth-12, a nonionic ethoxylated alcohol composed of fatty alcohols with 12 to 15 carbon atoms reacted with approximately 12 moles of ethylene oxide. This emulsifier is commonly used in personal care formulations for its ability to provide mild emulsification and a smooth skin or hair feel. In some embodiments, these emulsifiers may be combined to achieve synergistic effects, optimizing emulsion texture, viscosity, and product aesthetics. The inclusion of such emulsifiers may also improve the deposition of actives, enhance rinse-off behavior, and contribute to the overall sensorial profile of the product. The choice and concentration of emulsifier(s) may be selected based on factors such as formulation type (e.g., spray, foam, gel), phase polarity, ingredient solubility, and the desired final appearance and stability of the hair styling product.

In some embodiments, the additive may be a conditioner such as Crotein™ ADW (AMP-isostearoyl hydrolyzed (modified) wheat protein available from Croda Inc.). In some embodiments the additive may impact hair growth such as, for example, Rhodiola extract.

In some embodiments, the dry shampoo compositions may include at least one thickening or rheology agent to enhance the rheological properties, formulation stability, and application aesthetics of the product. While carbomer is a commonly employed synthetic thickener due to its high efficiency and clarity in aqueous systems, alternative thickeners may be utilized to achieve similar or improved performance characteristics, particularly in response to formulation constraints or desired sensory profiles. Suitable alternative synthetic or semi-synthetic thickeners include acrylates/C10-30 alkyl acrylate crosspolymer, which provides excellent viscosity enhancement and emulsion stabilization upon neutralization, as well as polyacrylamide and ammonium acryloyldimethyltaurate/VP copolymer, both of which offer enhanced texture and salt tolerance. In further embodiments, the composition may incorporate naturally derived or bio-based thickeners. Examples include xanthan gum, guar gum, sclerotium gum, gellan gum, and carrageenan, which are polysaccharide thickeners derived from microbial fermentation or botanical sources. The choice of thickening agent may be guided by considerations such as desired formulation transparency, pH compatibility, electrolyte tolerance, processing method, and the target sensory characteristics of the hair styling product.

In some embodiments, the dry shampoo compositions may include a softener, suspending agent, a masking agent (e.g., zeolite), an oil absorber, a friction agent, a UV protection component, or filter (e.g., Benzophenone-4), or damage prevention additive (e.g., sunflower oil).

Other suitable additive components will be known to those of ordinary skill in the art. Non-limiting examples of additive components include: antioxidants; essential oils; waxes; fillers; hair-fixative polymers; deodorizing agents; pediculicides; anti-dandruff agents; cosmetic and/or dermatological active agents including moisturizers, vitamins and their derivatives, essential fatty acids, sunscreens, herb and/or plant extracts; dispersing or suspending agents; anti-static agents; pearlescent aids; opacifiers; coloring agents, pigments (e.g., Umber Iron Oxide-earth pigment of iron oxide+manganese oxide+clay), or dyes (e.g., iron oxide); odor neutralizers (e.g., charcoal, ordenone); and sequestering agents; or combinations thereof. In some embodiments, the additive component may be capable of and/or may aid as a suspending agent, a dispersing agent, and/or a cleaning agent.

In some embodiments, the dry shampoo compositions optionally includes about 0.01% to about 5.0% by weight of the at least one additive. For example, the dry shampoo compositions may include less than about 5%, less than about 4%, less than about 3%, less than about 2.5%, less than about 2%, less than about 1.5%, less than about 1%, about 0% to about 2.5%, about 0% to about 2%, about 0% to about 1.5%, about 0% to about 1%, about 0.1% to about 2.5%, about 0.1% to about 2%, about 0.1% to about 1.5%, or about 0.1% to about 1% by weight of the at least one additive. In some embodiments, the dry shampoo compositions include about 0.1%, about 0.2%, about 0.3%, about 0.4%, about 0.5%, about 0.6%, about 0.7%, about 0.8%, about 0.9%, or about 1.0% by weight of the at least one additive.

In some embodiments, the dry shampoo compositions does not include a hydrofluorocarbon (HFC), a chlorofluorocarbon (CFC), or a hydrochlorofluorocarbon (HCFC). In certain embodiments, the dry shampoo compositions do not include a fluorocarbon (e.g., a hydrofluorocarbon (HFC)). For example, the dry shampoo compositions may be free of a fluorocarbon (e.g., 1,1 difluoroethane (152a) or 1,1,1,2 tetrafluoroethane (134a).

### Products and Methods

The present disclosure relates to dry shampoo compositions that may be used for topical application to hair (e.g., human hair) when the hair is in need to cleaning without the need for water. The dry shampoo compositions provided herein may be formulated as a spray or powder. The spray may be under pressure so as to propel the dry shampoo composition onto the hair upon engagement. The dry shampoo compositions provided herein may function, in part, by absorbing excess oil and sebum from the scalp and hair, thereby reducing the appearance of greasiness and extending the time between conventional washes.

The dry shampoo compositions provided herein deliver considerable convenience and versatility, particularly for individuals with limited access to water or those requiring rapid grooming solutions. The dry shampoo compositions provided herein may be subject to repeated use but avoid buildup on the scalp or hair shaft thereby avoiding irritation or obstruction of hair follicles. The dry shampoo compositions provided herein avoid noticeable residue, especially on darker hair. Finally, the dry shampoo compositions provided herein meet or exceed regulatory and safety requirements relevant to dry shampoos.

In one embodiment, the dry shampoo composition is suitable to be dispensed via a Bag-on-Valve (BOV) pump system, a type of pressurized dispensing technology commonly employed in aerosol-like containers. The dry shampoo compositions as provided herein may be contained in a sealed bag attached to a valve while propellant is placed outside the sealed bag (i.e., between the bag and the can). The Bag-on-Valve system may utilize compressed gas, such as nitrogen gas, as the propellant, which is non-flammable, inert, and environmentally friendly. The dry shampoo composition may be housed within the sealed, flexible bag which is typically constructed from laminated foil or plastic and positioned inside the can. This configuration allows for 360-degree application and minimizes dry shampoo composition waste by enabling nearly complete evacuation of the contents.

The BOV-pump dry shampoo system may operate by maintaining a pressurized gas environment between the inner pouch and the can wall. When the actuator is engaged, the pressure from the nitrogen compresses the pouch, thereby cleanly dispensing the product without mixing it with the propellant. This separation between the propellant and the formulation helps to preserve the chemical and physical integrity of the dry shampoo composition, making the system particularly advantageous for formulations that are sensitive, highly viscous, or free from preservatives.

In some embodiments, the dry shampoo compositions provided herein may be adapted to and delivered as an aerosol (e.g., include aerosol dry shampoo compositions). In some embodiments, the disclosure relates to a dry shampoo product that includes a container, a dry shampoo composition located in an interior product storage region of the container, and a spraying device. The spraying device may include a stem orifice, an actuator orifice, and optionally a vapor tap orifice. In some embodiments, the dry shampoo product may include a container, a dry shampoo composition located in an interior product storage region of the container, and a spraying device that includes a stem, an actuator, and a housing. According to one embodiment, the dry shampoo composition is stored directly with a gas (e.g., nitrogen gas) in the container. The container may be pressurized with the compressed gas and, as an actuator is pressed, the compressed gas pushes the dry shampoo composition out.

In some embodiments, the housing of the spraying device includes a vapor tap orifice. In certain cases, the vapor tap orifice of the spraying device is about 0.010 to about 0.062 inches in diameter. In some embodiments, the housing of the spraying device includes a stem orifice that is about 0.010 inches to about 0.040 inches in diameter.

In some embodiments, the spraying device is connected to the container by a crimp. In certain cases, the crimp has a depth of about 0.195 inches to about 0.205 inches. In certain cases, the crimp has a depth of up to about 1.10 inch. The crimp may have a diameter of about 1.075 inches to about 1.65 inches. The crimp may have a height of up to about 0.3 inches.

In some embodiments, the container includes a head space of about 15.0% to about 50.0% (e.g., about 20.0% to about 35.0%).

In some embodiments, the container including a dry shampoo composition has a pressure (e.g., at a temperature of about 25° C.) of about 50.0 psi to about 150.0 psi, about 75.0 psi to about 140.0 psi, about 95.0 psi to about 135.0 psi, or about 100.0 psi to about 130.0 psi.

In some embodiments, the spraying device delivers the dry shampoo composition at a rate of at least about 0.25 g/s (e.g., about 0.25 g/s to about 2.00 g/s). The spraying device may deliver the dry shampoo composition at a rate of about 0.25 g/s to about 1.8 g/s, about 0.25 g/s to about 1.6 g/s, about 0.25 g/s to about 1.4 g/s, about 0.25 g/s to about 1.2 g/s, about 0.25 g/s to about 1.0 g/s, about 0.4 g/s to about 1.2 g/s, about 0.5 g/s to about 1.8 g/s, about 0.5 g/s to about 1.6 g/s, about 0.5 g/s to about 1.4 g/s, about 0.5 g/s to about 1.2 g/s, about 0.5 g/s to about 1.0 g/s, about 0.75 g/s to about 1.8 g/s, about 0.75 g/s to about 1.6 g/s, about 0.75 g/s to about 1.4 g/s, about 0.75 g/s to about 1.2 g/s, or about 0.75 g/s to about 1.0 g/s. In some embodiments, the spraying device delivers the dry shampoo composition with a spray diameter of about 1.5 inches to about 3.0 inches at a distance of about 6 inches.

In some embodiments, the spraying device includes an average spray cone angle of from about 25 degrees to about 35 degrees. In some embodiments, the spraying device includes an exit orifice of from about 0.18 mm to about 0.3 mm.

In some embodiments, the dry shampoo compositions provided herein may be delivered via a mechanical sprayer. In such embodiments, the dry shampoo composition is in liquid form an forced through a nozzle and out as a spray or mist when a pump actuator is pressed to build pressure.

It should be understood, however, that a dry shampoo composition may also be provided using alternative delivery methods, such as applying by hand directly to the hair or using a container fitted with a pump to dispense a liquid composition (e.g., atomizer), as will be understood by those of ordinary skill in the art.

Method for cleaning hair are also provided. According to one embodiment, the method of cleaning hair includes the step of introducing the dry shampoo composition to the scalp or hair of a subject in need thereof to reduce oiliness and refresh the hair without rinsing. After introduction, the cleaned hair may be styled. According to one embodiment, the dry shampoo composition is ejected by a product including a container and a spraying device suitable for ejecting the dry shampoo composition. According to one embodiment, the introduction step includes dispensing the product onto the roots and scalp of dry hair, allowing the dry shampoo composition to sit for a brief period to maximize oil absorption, and then distributing the composition by brushing or massaging through the hair. This process not only helps eliminate visible oil but can also impart additional volume and texture to the hair.

### EXEMPLARY EMBODIMENTS

A potential feature of a dry shampoo composition according to exemplary embodiments of the present disclosure is that the composition provides high oil-absorption. A dry shampoo composition, according to exemplary embodiments of the present disclosure, satisfies a long-felt need for a product that includes many, if not all, of the following features: low environmental impact, improved oil-absorption, and improved hair appearance after use.

In some embodiments, the disclosure relates to compositions and products relating to a dry shampoo composition. Such compositions may reduce or eliminate an oily and/or unaesthetic appearance of hair.

In some embodiments, the present disclosure provides a dry shampoo composition that includes about 75.0% to about 85.0% by weight of a propellant, about 1.0% to about 15.0% by weight of an oil-absorbing component, about 5.0% to about 20.0% by weight of a solvent, and optionally up to about 2.5% of one or more additives.

In some embodiments, the present disclosure provides a dry shampoo composition that includes about 50.0% to about 90.0% by weight of a propellant, about 1.0% to about 20.0% by weight of an oil-absorbing component, about 5.0% to about 40.0% by weight of a solvent, and optionally up to about 5.0% of one or more additives.

In some embodiments, the present disclosure provides a dry shampoo composition that includes about 30.0% to about 90.0% by weight of a first propellant, 30.0% to about 90.0% by weight of a second propellant, about 1.0% to about 20.0% by weight of an oil-absorbing component, about 5.0% to about 40.0% by weight of a solvent, and optionally up to about 25.0% of one or more additives.

In certain embodiments, the dry shampoo composition conforms to the California Code of Regulations, Title 17, Section 94511 (c). For example, the dry shampoo composition (e.g., “Innovative Liquefied Propellant Product”) meets all of the following criteria:
(A) For a product manufactured before Jan. 1, 2029, the Innovative Liquefied Propellant Product achieves a 50 percent or greater reduction in GHG emissions compared to the Representative HFC-152a Product;
(B) For a product manufactured on or after Jan. 1, 2029, the Innovative Liquefied Propellant Product includes only ingredients with a GWP less than 10, or achieves a 90 percent or greater reduction in GHG emissions compared to the Representative HFC-152a Product; and
(C) The ozone-forming potential of the Innovative Liquefied Propellant Product does not exceed that of the Representative HFC-152a Product.

In certain embodiments, the dry shampoo composition has a maximum incremental reactivity (MIR) of equal to or less than the MIR of a representative HFC-152a product formulation of a dry shampoo (55% VOC) provided by the California Air Resources Board (CARB).

In certain embodiments, the dry shampoo composition has a maximum incremental reactivity (MIR) of equal to or less than about 0.7540. For example, the dry shampoo composition may have an MIR of less than about 0.80, less than about 0.78, less than about 0.76, about 0.50 to about 0.80, about 0.50 to about 0.78, or about 0.50 to about 0.76.

In certain embodiments, the dry shampoo composition includes only components with a Global Warming Potential (GWP) of less than 10. For example, the dry shampoo composition includes only components with a Global Warming Potential (GWP) of less than about 10, less than about 5, or less than about 2. In certain embodiments, the dry shampoo composition includes only components with a Global Warming Potential (GWP) of 0.

In certain embodiments, the dry shampoo composition has a Global Warming Potential (GWP) of equal to or less than 150.

In certain embodiments, the dry shampoo composition has a 50% or greater (e.g., a 90% or greater) reduction in greenhouse gas (GHG) emissions compared to a representative HFC-152a product formulation of a dry shampoo (55% VOC) provided by the California Air Resources Board (CARB).

In some embodiments, the dry shampoo composition includes a weight ratio of oil absorbing powder to propellant of about 1 to 2 to about 1 to 20. For example, the dry shampoo composition may include a weight ratio of oil absorbing powder to solvent of about 1 to 5 to about 1 to 15, or about 1 to 5 to about 1 to 10. In some embodiments, the dry shampoo composition includes a weight ratio of oil absorbing powder to solvent of about 1 to 5, about 1 to 6, about 1 to 7, about 1 to 8, about 1 to 9, or about 1 to 10.

In some embodiments, the dry shampoo composition includes a weight ratio of oil absorbing powder to solvent of about 2 to 1 to about 1 to 2. For example, the dry shampoo composition may include a weight ratio of oil absorbing powder to solvent of about 1.2 to 1, about 1.1 to 1, about 1 to 1, about 1 to 1.1, or about 1 to 1.2.

In some embodiments, the dry shampoo composition includes a weight ratio of oil absorbing powder to solvent of about 1 to 2 to about 1 to 50. For example, the dry shampoo composition may includes a weight ratio of oil absorbing powder to solvent of about 1 to 3, about 1 to 4, about 1 to 5, about 1 to 6, about 1 to 7, about 1 to 8, about 1 to 9, about 1 to 10, about 1 to 15, about 1 to 20, or about 1 to 25.

In some embodiments, the dry shampoo composition includes about 10% to about 25% by weight of an oil absorbing powder and an alcohol. For example, the dry shampoo composition may include about 10% to about 20%, about 10% to about 15%, about 15% to about 25%, about 15% to about 24%, about 15% to about 23%, about 15% to about 22%, about 15% to about 21%, about 15% to about 20%, about 16% to about 25%, about 17% to about 25%, about 18% to about 25%, about 19% to about 25%, about 16% to about 24%, about 17.5% to about 22.5%, or about 18% to about 22% by weight of an oil absorbing powder and an alcohol. In some embodiments, the dry shampoo composition includes about 10%, about 11%, about 12%, about 13%, about 14%, about 15%, about 16%, about 17%, about 18%, about 19%, about 20%, about 21%, about 22%, about 23%, about 24%, or about 25% by weight of an oil absorbing powder and an alcohol.

In some embodiments, the dry shampoo composition does not include water. For example, the dry shampoo composition may be substantially free of water. In some embodiments, the dry shampoo composition includes less than about 5% by weight of water. For example, the dry shampoo composition may include less than about 2% or less than about 1% by weight of water.

In some embodiments, the dry shampoo composition does not include a hydrofluorocarbon (HFC), a chlorofluorocarbon (CFC), or a hydrochlorofluorocarbon (HCFC). In certain embodiments, the dry shampoo composition does not include a fluorocarbon (e.g., a hydrofluorocarbon (HFC)). For example, the dry shampoo composition may be substantially free of a fluorocarbon (e.g., 1,1 difluoroethane (152a) or 1,1,1,2 tetrafluoroethane (134a).

In some embodiments, the disclosure relates to a dry shampoo composition including: about 75.0% to about 85.0% by weight of at least one propellant; about 7.5% to about 12.5% by weight of an alcohol (e.g., isopropanol); about 7.5% to about 12.5% by weight of a starch (e.g., rice starch); about 0.01% to about 0.5% by weight of a silica; and optionally up to about 5% of an additive. In some embodiments, the dry shampoo composition includes about 75% to about 85% by weight of at least one propellant; about 7.5% to about 12.5% by weight of isopropanol; about 7.5% to about 12.5% by weight of a rice starch; about 0.01% to about 0.5% by weight of a silica; about 0.01% to about 0.5% by weight of isopropyl palmitate; and about 0.01% to about 0.5% by weight of a fragrance.

In some embodiments, the disclosure relates to a dry shampoo composition including: about 75.0% to about 85.0% by weight of a propellant (e.g., an ether); about 7.5% to about 12.5% by weight of an alcohol (e.g., isopropanol); about 7.5% to about 12.5% by weight of a starch (e.g., rice starch); and optionally up to about 5% of an additive. In some embodiments, the dry shampoo composition includes about 75% to about 85% by weight of dimethyl ether; about 7.5% to about 12.5% by weight of propanol; about 7.5% to about 12.5% by weight of a rice starch; about 0.01% to about 0.5% by weight of a silica; about 0.01% to about 0.5% by weight of isopropyl palmitate; and about 0.01% to about 0.5% by weight of a fragrance.

In some embodiments, the disclosure relates to a dry shampoo composition including: about 70.0% to about 85.0% dimethyl ether; about 5.0% to about 15.0% isopropyl alcohol; about 8.0% to about 12.0% rice starch (e.g., Oryza sativa (rice) starch); about 0.1% to about 1.0% silica; about 0.05% to about 0.5% isopropyl palmitate; and about 0.05% to about 1.0% fragrance. Such a dry shampoo may be completely free or substantially free of talc and water and exhibits a MIR of less than 0.72.

In some embodiments, the disclosure relates to a dry shampoo composition including: about 50.0% to about 70.0% dimethyl ether; about 15.0% to about 30.0% water; about 5.0% to about 15.0% ethanol; about 0.1% to about 2.0% of a preservative including phenoxyethanol and ethylhexylglycerin; about 0.005% to about 0.1% of a polymeric film-former that includes PVM/MA decadiene crosspolymer; about 0.001% to about 0.1% of a pH adjusting agent that includes sodium hydroxide; about 0.1% to about 1.0% glycerin; about 0.5% to about 2.0% propanediol; about 0.05% to about 0.5% tetrasodium glutamate diacetate; about 0.5% to about 2.0% of a hydrophobic emollient that includes C13-15 alkane and heptyl undecylenate; about 0.1% to about 1.0% terpinolene; and about 2.0% to about 6.0% of an oil-absorbing component. Such a dry shampoo may be completely free or substantially free of talc and water and exhibits a MIR of less than 0.72. The oil-absorbing powder may include silica with a median particle size of less than 10 microns; and silica with a spherical morphology and oil absorption capacity of at least 100 mL/100 g, 200 mL/100 g, 300 mL/100 g or more.

In some embodiments, the disclosure relates to a dry shampoo composition including: about 30.0% to about 90.0% by weight of a first propellant; about 30.0% to about 90.0% by weight of a second propellant; about 5.0% to about 40.0% by weight of an alcohol (e.g., isopropanol); about 1.0% to about 20.0% by weight of a starch (e.g., rice starch); about 0.01% to about 5.0% by weight of a silica; up to about 0.5% by weight of isopropyl palmitate; and up to about 5.0% by weight of at least one fragrance. According to such an embodiment, the MIR is less than about 0.73.

In some embodiments, the disclosure relates to a dry shampoo composition including: about 30.0% to about 90.0% by weight of a first propellant; about 30.0% to about 90.0% by weight of a second propellant; about 5.0% to about 40.0% by weight of an alcohol (e.g., isopropanol); about 1.0% to about 25.0% by weight of a sunscreen component; about 1.0% to about 20.0% by weight of a starch (e.g., rice starch); up to about 5.0% by weight diisopropyl adipate (Schercemol DIA ester); up to about 5.0% by weight hectorite clay; up to about 5.0% by weight Umber Iron Oxide; up to about 5.0% by weight of at least one fragrance; and up to about 5.0% by weight sea buckthorn-derived omega-rich nutraceutical. According to such an embodiment, the MIR is less than about 0.70.

In some embodiments, the disclosure relates to a dry shampoo composition including: about 30.0% to about 90.0% by weight of a first propellant; about 30.0% to about 90.0% by weight of a second propellant; about 5.0% to about 40.0% by weight of an alcohol (e.g., isopropanol); about 1.0% to about 20.0% by weight of a starch (e.g., rice starch); up to about 5.0% by weight hectorite clay; up to about 5.0% by weight propanediol; up to about 5.0% by weight glycerine; up to about 5.0% by weight panthenol; up to about 5.0% by weight Avobenzone USP (butyl methoxydibenzoylmethane); up to about 15.0% by weight Parsol HMS (3,3,5-Trimethylcyclohexyl salicylate); up to about 5.0% by weight Parsol EHS (ethylhexyl salicylate); and up to about 10.0% by weight octocrylene.

According to such an embodiment, the MIR is less than about 0.74.

In some embodiments, the disclosure relates to a dry shampoo composition including: about 30.0% to about 90.0% by weight of a first propellant; about 30.0% to about 90.0% by weight of a second propellant; about 5.0% to about 40.0% by weight of at least one alcohol; about 1.0% to about 20.0% by weight of at least one starch; up to about 5.0% by weight of a citric acid; up to about 5.0% weight of lactic acid; up to about 5.0% by weight hectorite clay; up to about 5.0% by weight of at least one fragrance; and up to about 5.0% by weight of at least one chelating agent that includes from about 35.0 wt % to about 63.9 wt % sodium polyitaconate, about 35.0 wt % to about 61.0 wt % sodium citrate and about 0.10 wt % to about 5.0 wt % Sapindus mukorossi fruit extract. According to such an embodiment, the MIR is less than about 0.74.

In some embodiments, the disclosure relates to a dry shampoo composition including: about 30.0% to about 90.0% by weight of a first propellant; about 30.0% to about 90.0% by weight of a second propellant; about 5.0% to about 40.0% by weight of an alcohol; about 1.0% to about 20.0% by weight of a starch; up to about 5.0% by weight of isopropyl isostearate; up to about 5.0% by weight of sea buckthorn seed oil; up to about 5.0% by weight of at least one odor neutralizer, up to about 5.0% by weight hectorite clay; and up to about 5.0% by weight of at least one fragrance. According to such an embodiment, the MIR is less than about 0.74.

### EXAMPLES

The examples and preparations provided below further illustrate and exemplify particular aspects of embodiments of the disclosure. It is to be understood that the scope of the present disclosure is not limited in any way by the scope of the following examples.

### Example 1

An exemplary dry shampoo in accordance with certain aspects of the present disclosure is provided in Table 1. The MIR was 0.73. Each component of the dry shampoo composition had a GWP of 0. Thus, the dry shampoo composition provided a GHG emission of 0.

TABLE 1
Dry Shampoo Composition

| Component | Function | wt % |
|---|---|---|
| BUTANE | Propellant | 40.00 |
| PROPANE | Propellant | 40.00 |
| ISOPROPYL ALCOHOL | Solvent | 9.38 |
| ORYZA SATIVA (RICE) STARCH WITH | Oil-Absorbing Component | 10.00 |
| CETRIMONIUM CHLORIDE | | |
| SILICA (AEROSIL 200 F) | Oil-Absorbing Component | 0.25 |
| ISOPROPYL PALMITATE | Emollient | 0.19 |
| FRAGRANCE (PARFUM) | Fragrance | 0.19 |

### Example 2

An exemplary formulation in accordance with certain aspects of the present disclosure is in Table 2. The MIR was 0.72. Each component had a GWP of 0. Thus, the dry shampoo composition provided a GHG emission of 0.

TABLE 2
Dry Shampoo Composition

| Name | Function | wt % |
|---|---|---|
| DIMETHYL ETHER | Propellant | 80.00 |
| ISOPROPYL ALCOHOL | Solvent | 9.38 |
| ORYZA SATIVA (RICE) STARCH | Oil-Absorbing Component | 10.00 |
| WITH CETRIMONIUM CHLORIDE | | |
| SILICA (AEROSIL 200 F) | Oil-Absorbing Component | 0.25 |
| Dry Shampoo Composition | | |
| ISOPROPYL PALMITATE | Emollient | 0.19 |
| FRAGRANCE (PARFUM) | Fragrance | 0.19 |

### Example 3

An exemplary formulation in accordance with certain aspects of the present disclosure is provided in Table 3. The MIR was 0.73. Each component had a GWP of 0. Thus, the dry shampoo composition provided a GHG emission of 0.

TABLE 3
Dry Shampoo Composition

| Name | Function | wt % |
|---|---|---|
| DIMETHYL ETHER (DME) | Propellant | 60.0 |
| WATER | Solvent | 22.02 |
| ETHANOL | Solvent | 10.00 |
| PHENOXYETHANOL & | Preservative | 0.40 |
| ETHYLHEXYLGLYCERIN | | |
| PVM/MA DECADIENE CROSSPOLYMER | Hair Fixative | 0.02 |
| SODIUM HYDROXIDE | Buffering Agent | 0.01 |
| GLYCERIN | Emollient | 0.20 |
| PROPANEDIOL | Emollient | 1.00 |
| TETRASODIUM GLUTAMATE ACETATE | Chelating Agent | 0.10 |
| C13-15 ALKANE (AND) HEPTYL | Emollient | 1.00 |
| UNDECYLENATE | | |
| TERPINOLENE | Fragrance | 0.75 |
| SILICA (SPHERON P-1500) | Oil-Absorbing | 4.00 |
| | Component | |
| SILICA (SOLESPHERE H-53) | Oil-Absorbing | 0.50 |
| | Component | |

The dry shampoo compositions of Examples 2 and 3 combined effective oil absorption, rapid drying, and environmentally responsible design. Unlike many conventional aerosol dry shampoos, these exemplary dry shampoo compositions achieved excellent hair-refreshing performance without relying on components with high Global Warming Potential (GWP) or excessive volatile organic compound (VOC) content. The dry shampoo composition of Example 2 combined prominent levels of dimethyl ether (DME), a low MIR and low-GWP propellant, with a blend of Oryza sativa (rice) starch (with cetrimonium chloride), and AEROSIL 200 F silica (hydrophilic fumed silica with a specific surface area of from about 175 m²/g to about 200 m²/g). This silica imparted robust oil-absorbing capacity, volumizing texture, and residue-free finish. The inclusion of cetrimonium chloride, a quaternary ammonium compound, enhanced deposition on hair fibers while aiding in the even dispersion of starch particles. The formulation also demonstrated fast drying, attributed to the high propellant content and absence of high-moisture solvents, providing a lightweight and clean-feeling result on hair. Notably, the MIR of 0.72 and GWP of 0 confirm this formulation's superior environmental profile compared to prior art compositions containing hydrofluorocarbons (HFCs) or alkanes.

The dry shampoo of Example 3, while incorporating water and ethanol to improve solubility and usability in certain systems, still maintained a MIR of 0.73 and zero GWP through careful selection of solvents and additives. The combination of Spheron P-1500 (synthetic, highly porous, amorphous silica) and Solesphere H-53 (amorphous silica (microspherical silica gel); oil absorption capacity (up to 400 mL per 100 g) silicas provided a complementary oil-absorption profile that enhanced performance even in the presence of a small water fraction. The inclusion of additional conditioning and performance-enhancing agents such as propanediol, glycerin, and PVM/MA Decadiene Crosspolymer contributed to hair feel, scalp compatibility, and texture. Despite a slightly higher MIR than Example 2, the dry shampoo composition of Example 3 still represented a marked improvement over comparative formulations (provided herein) in both environmental impact and cosmetic performance.

### Comparative Example 1

A comparative dry shampoo composition is provided in Table 4 (referred to as Comparative Dry Shampoo Composition #1). The MIR of Comparative Dry Shampoo Composition #1 was 0.72. Comparative Dry Shampoo Composition #1 provided inferior performance with respect to long drying time, minimal oil-absorbing, and high humidity. The inferior performance of Comparative Dry Shampoo Composition #1 may be the result of proportionally prominent levels of water (i.e., moisture excess). Each component of Comparative Dry Shampoo Composition #1 had a GWP of 0. Thus, Comparative Dry Shampoo Composition #1 provided a GHG emission of 0.

TABLE 4
Comparative Dry Shampoo Composition #1

| Name | Function | wt % |
|---|---|---|
| ALKANE BLEND (AB70) | Propellant | 76.796 |
| ISOPROPYL ALCOHOL | Solvent | 2.79 |
| ETHANOL | Solvent | 0.0015 |
| DI WATER | Solvent | 10.00 |
| TAPIOCA STARCH | Oil Absorbing Powder | 10.00 |
| PROPANEDIOL | Emollient | 0.12 |
| GLYCERIN | Emollient | 0.058 |
| PANTHENOL | Emollient | 0.023 |
| MODIFIED WHEAT PROTEIN | Additive | 0.00084 |
| FRAGRANCE | Additive | 0.21 |

The Comparative Dry Shampoo Composition #1 is believed to have exhibited inferior technical performance due to its high water content and low propellant-to-solvent ratio, resulting in a slow-drying formulation that leaves the hair feeling wet and lacks adequate oil absorption. The inclusion of tapioca starch without synergistic dispersants such as quaternary surfactants is believed to have resulted in limited powder distribution and performance under humid conditions.

### Comparative Example 2

A comparative formulation is provided in Table 5 (referred to as Comparative Dry Shampoo Composition #2). The MIR of Comparative Dry Shampoo Composition #2 was 0.79. Comparative Dry Shampoo Composition #2 provided a GWP of 3844 per 100-gram product.

TABLE 5
Comparative Dry Shampoo Composition #2

| Name | Function | wt % |
|---|---|---|
| HFC 152a | Propellant | 31.00 |
| ISO-BUTANE | Propellant | 39.00 |
| ETHANOL | Solvent | 15.32 |
| RICE STARCH | Oil Absorbing Powder | 12.00 |
| MODIFIED STARCH (NATRASORB HFB) | Oil Absorbing Powder | 1.50 |
| PVP/VA COPOLYMER | Hair Fixative | 0.15 |
| FRAGRANCE | Fragrance | 1.00 |
| ETHYLHEXYL METHOXYCINNAMATE | Additive | 0.02 |
| PANTHENOL | Emollient | 0.003 |
| RHODIOLA EXTRACT | Additive | 0.003 |
| VITAMIN (BV-OSC) | Additive | 0.003 |

Comparative Dry Shampoo Composition #2 demonstrated an unacceptable environmental profile, with a GWP of 3844 per 100 g of product likely due to the inclusion of HFC-152a and isobutane. The environmental penalties and regulatory risks associated with high-GWP propellants are believed to undermine commercial viability in regions with strict VOC or climate legislation.

### Comparative Example 3

A comparative formulation is provided in Table 6 (referred to as Comparative Dry Shampoo Composition #3). The MIR of Comparative Dry Shampoo Composition #3 was 0.73. Comparative Dry Shampoo Composition #3 provided inferior performance with respect to long drying time, and minimal oil-absorbing. The inferior performance of Comparative Dry Shampoo Composition #3 may be the result of prominent levels of solvent (i.e., moisture excess). Comparative Dry Shampoo Composition #3 provided a GWP of 3596 per 100-gram product.

TABLE 6
Comparative Dry Shampoo Composition #3

| Name | Function | wt % |
|---|---|---|
| HFC 152a | Propellant | 29.00 |
| ALKANE BLEND (AB70) | Propellant | 24.00 |
| ETHANOL | Solvent | 30.59 |
| ISOHEXADECANE | Solvent | 4.626 |
| DI WATER | Solvent | 0.10 |
| RICE STARCH | Oil Absorbing Powder | 10.00 |
| DECAMETHYLCYCLOPENTASILOXANE | Conditioner | 1.00 |
| PROPANEDIOL | Emollient | 0.21 |
| GLYCERIN | Emollient | 0.12 |
| PANTHENOL | Emollient | 0.26 |
| MODIFIED WHEAT PROTEIN | Conditioner | 0.10 |
| FRAGRANCE | Fragrance | 0.0002 |

Comparative Dry Shampoo Composition #3 had a lower MIR than Comparative Dry Shampoo Composition #2 but still suffered from long drying times and moisture overload, due, in part, to excessive ethanol and inclusion of other slow-evaporating solvents such as isohexadecane and decamethylcyclopentasiloxane. Comparative Dry Shampoo Composition #3 also included multiple propellants and conditioning agents, but the performance trade-offs render it suboptimal for modern dry shampoo users seeking speed, lightness, and a clean finish.

### STATEMENTS OF THE DISCLOSURE

The following summary presents certain aspects of the present disclosure and is not intended to limit the scope of the invention as defined by the claims.
Statement 1. A dry shampoo composition, the composition including:
about 50.0 wt % to about 90.0 wt % of a liquefied gas propellant;
about 1.0 wt % to about 20.0 wt % of a concentrate, the concentrate comprising an oil-absorbing powder; and
about 5.0 wt % to about 40.0 wt % of a solvent (e.g., an alcohol or water),
wherein the composition has a combined maximum incremental reactivity (MIR) of less than about 0.76.
Statement 2. The composition of Statement 1, wherein the liquefied gas propellant includes an alkane, an ether, or a combination thereof.
Statement 3. The composition of Statement 1 or Statement 2, wherein the liquefied gas propellant includes an alkane selected from the group consisting of ethane, propane, butane (e.g., n-butane or iso-butane), pentane (e.g., n-pentane or isopentane), blends of hydrocarbons (e.g., C3-C7 hydrocarbons), and combinations thereof.
Statement 4. The composition of Statement 1, wherein the liquefied gas propellant includes an alkane blend, the alkane blend comprising a first alkane and a second alkane that is different than the first alkane.
Statement 5. The composition of Statement 1 or Statement 2, wherein the liquefied gas propellant includes an ether (e.g., dimethyl ether).
Statement 6. The composition of any one of the preceding statements, wherein the composition includes about 75.0 wt % to about 85.0 wt % of the liquefied gas propellant.
Statement 7. The composition of any one of the preceding statements, wherein the oil-absorbing powder includes a starch (e.g., potato starch, rice starch, tapioca starch, wheat starch, or corn starch).
Statement 8. The composition of any one of the preceding statements, wherein the oil-absorbing powder includes a silica.
Statement 9. The composition of any one of Statements 1 to 7, wherein the oil-absorbing powder includes a silica mixture, the silica mixture including a first silica and a second silica that is different than the first silica.
Statement 10. The composition of any one of the preceding statements, wherein the composition includes about 1.0 wt % to about 15.0 wt % by weight of the oil-absorbing powder.
Statement 11. The composition of any one of the preceding statements, wherein the solvent includes an alcohol selected from the group consisting of ethanol, propanol, butanol, and any combination thereof, preferably wherein the alcohol is isopropanol.
Statement 12. The composition of Statement 11, wherein the composition includes about 5.0 wt % to about 15.0 wt % by weight of the alcohol.
Statement 13. The composition of any one of the preceding statements, wherein the composition further includes an additive selected from the group consisting of an emollient, a fragrance, a hair fixative, a plasticizer, a humectant, a conditioner, a softener, a suspending agent, a masking agent, an oil absorber, a friction agent, a UV filter, a pH adjuster, and any combination thereof.
Statement 14. The composition of any one of the preceding statements, wherein the composition is substantially free of water.
Statement 15. The composition of any one of Statements 1 to 13, wherein the composition includes water.
Statement 16. The composition of any one of the preceding statements, wherein the composition is substantially free of a fluorocarbon (e.g., 1,1-difluoroethane (152a) or 1,1,1,2-tetrafluoroethane (134a)).
Statement 17. A dry shampoo composition, the composition including:
about 75.0 wt % to about 85.0 wt % by weight of an alkane blend (e.g., propane and butane);
about 7.5 wt % to about 12.5 wt % by weight of propanol (e.g., isopropanol);
about 7.5 wt % to about 12.5 wt % by weight of a rice starch; and
about 0.01 wt % to about 0.5 wt % by weight of a silica;
wherein the composition has a combined maximum incremental reactivity (MIR) of about 0.50 to about 0.76.
Statement 18. A dry shampoo composition, the composition including:
about 75.0 wt % to about 85.0 wt % dimethyl ether;
about 7.5 wt % to about 12.5 wt % propanol (e.g., isopropanol);
about 7.5 wt % to about 12.5 wt % rice starch; and
about 0.01 wt % to about 0.5 wt % silica;
wherein the composition has a combined maximum incremental reactivity (MIR) of about 0.50 to about 0.76.
Statement 19. A dry shampoo composition, the composition including:
about 55.0 wt % to about 65.0 wt % dimethyl ether;
about 15.0 wt % to about 25.0 wt % water;
about 7.5 wt % to about 12.5 wt % ethanol; and
about 0.1 wt % to about 7.5 wt % of a first silica and a second silica that is different from the first silica;
wherein the composition has a combined maximum incremental reactivity (MIR) of about 0.50 to about 0.76.
Statement 20. A dry shampoo product including:
a container;
a dry shampoo composition according to any one of Statements 1 through 19 located in an interior product storage region of the container; and
a spraying device comprising a stem orifice, an actuator orifice, a body orifice of about 0.010 inches to about 0.062 inches and optionally a vapor tap orifice.
Statement 21. The dry shampoo product of Statement 20, wherein the stem orifice is about 0.010 inches to about 0.040 inches in diameter.
Statement 22. The dry shampoo product of Statement 20 or Statement 21, wherein the actuator orifice is about 0.010 inches to about 0.030 inches in diameter.
Statement 23. The dry shampoo product of any one of Statements 20 to 22, wherein the vapor tap orifice is about 0.010 inches to about 0.062 inches in diameter.
Statement 24. The dry shampoo product of any one of Statements 20 to 23, wherein the spraying device delivers the dry shampoo composition at a rate of about 0.50 g/s to about 2.0 g/s.
Statement 25. A dry shampoo composition comprising by weight percent of the total composition:
about 70.0 wt % to about 85.0 wt % dimethyl ether;
about 5.0 wt % to about 15.0 wt % isopropyl alcohol;
about 8.0 wt % to about 12.0 wt % rice starch;
about 0.1 wt % to about 1.0 wt % silica;
about 0.05 wt % to about 0.5 wt % isopropyl palmitate; and
optionally, about 0.05 wt % to about 1.0 wt % fragrance,
wherein the composition is substantially free of talc and water; and
wherein the composition exhibits a maximum incremental reactivity (MIR) value of less than 0.72.
Statement 26. The composition of Statement 25, wherein the dimethyl ether is present in an amount of about 78.0% to about 82.0% by weight.
Statement 27. The composition of Statement 25, further comprising cetrimonium chloride in an amount of about 9.0% to about 11.0% by weight.
Statement 28. The composition of Statement 25, wherein the isopropyl alcohol is present in an amount of about 9.0% to about 10.0% by weight.
Statement 29. The composition of Statement 25, wherein the silica includes fumed silica in an amount of about 0.2% to about 0.3% by weight.
Statement 30. The composition of Statement 25, wherein the silica includes a spherical morphology and an oil absorption capacity of at least 200 mL/100 g.
Statement 31. The composition of Statement 25, wherein the propellant includes only dimethyl ether and has a global warming potential (GWP) of less than 2.
Statement 32. The composition of Statement 25, wherein the composition is dispensed via a spraying device comprising a vapor tap orifice having a diameter of about 0.010 inches to about 0.040 inches and a spray rate of about 0.75 g/s to about 1.2 g/s.
Statement 33. A dry shampoo composition comprising by weight percent of the total composition:
about 50.0 wt % to about 70.0 wt % dimethyl ether;
about 15.0 wt % to about 30.0 wt % water;
about 5.0 wt % to about 15.0 wt % ethanol;
about 0.1 wt % to about 2.0 wt % a preservative comprising phenoxyethanol and ethylhexylglycerin;
about 0.005 wt % to about 0.1 wt % a polymeric film-former comprising PVM/MA decadiene crosspolymer;
about 0.001 wt % to about 0.1 wt % a pH adjusting agent comprising sodium hydroxide;
about 0.1 wt % to about 1.0 wt % glycerin;
about 0.5 wt % to about 2.0 wt % propanediol;
about 0.05 wt % to about 0.5 wt % tetrasodium glutamate diacetate;
about 0.5 wt % to about 2.0 wt % an emollient comprising C13-15 alkane and heptyl undecylenate;
about 0.1 wt % to about 1.0 wt % terpinolene;
about 2.0 wt % to about 6.0 wt % an oil-absorbing component,
wherein the composition exhibits a maximum incremental reactivity (MIR) value of less than 0.72.
Statement 34. The composition of Statement 33, wherein the oil-absorbing component includes: silica with a median particle size of less than 10 microns; and silica with a spherical morphology and oil absorption capacity of at least 200 mL/100 g.
Statement 35. The composition of Statement 33, wherein the composition is dispensed via a spraying device comprising a vapor tap orifice having a diameter of about 0.010 inches to about 0.040 inches.
Statement 36. The composition of Statement 35, wherein the spraying device delivers the composition at a rate of about 0.75 g/s to about 1.2 g/s.
Statement 37. The composition of Statement 33, wherein the composition includes from about 20.0 wt % to about 25.0 wt % water as a solvent.
Statement 38. The composition of Statement 33, wherein the composition includes from about 5.0 wt % to about 15.0 wt % ethanol as a secondary solvent.
Statement 39. The composition of Statement 33, wherein the composition includes from about 0.30 wt % to about 0.50 wt % of a preservative selected from the group consisting of phenoxyethanol and ethylhexylglycerin.
Statement 40. The composition of Statement 33, wherein the oil-absorbing component includes from about 3.0 wt % to about 5.0 wt % spherical silica.
Statement 41. The composition of Statement 33, wherein the oil-absorbing component further includes from about 0.3 wt % to about 0.7 wt % porous silica.
Statement 42. The composition of Statement 33, wherein the emollient further includes from about 0.5 wt % to about 2.5 wt % of a mixture of propanediol and C13-15 alkane (and) heptyl undecylenate.
Statement 43. A method of refreshing or cleansing hair without water, the method including:
providing a dry shampoo composition; and
introducing or dispensing the dry shampoo composition to the scalp or hair of a subject in need thereof to reduce oiliness and refresh the hair without rinsing, wherein the dry shampoo composition includes:
   about 70.0 wt % to about 85.0 wt % dimethyl ether;
   about 5.0 wt % to about 15.0 wt % isopropyl alcohol;
   about 8.0 wt % to about 12.0 wt % rice starch;
   about 0.1 wt % to about 1.0 wt % silica;
   about 0.05 wt % to about 0.5 wt % isopropyl palmitate; and
   optionally, about 0.05 wt % to about 1.0 wt % fragrance,
   wherein the composition is substantially free of talc and water, and
   wherein the composition exhibits a maximum incremental reactivity (MIR) value of less than 0.72; or
wherein the dry shampoo composition includes:
   about 50.0 wt % to about 70.0 wt % dimethyl ether;
   about 15.0 wt % to about 30.0 wt % water;
   about 5.0 wt % to about 15.0 wt % ethanol;
   about 0.1 wt % to about 2.0 wt % a preservative comprising phenoxyethanol and ethylhexylglycerin;
   about 0.005 wt % to about 0.1 wt % a polymeric film-former comprising PVM/MA decadiene crosspolymer;
   about 0.001 wt % to about 0.1 wt % a pH adjusting agent comprising sodium hydroxide;
   about 0.1 wt % to about 1.0 wt % glycerin;
   about 0.5 wt % to about 2.0 wt % propanediol;
   about 0.05 wt % to about 0.5 wt % tetrasodium glutamate diacetate;
   about 0.5 wt % to about 2.0 wt % a hydrophobic emollient comprising C13-15 alkane and heptyl undecylenate;
   about 0.1 wt % to about 1.0 wt % terpinolene;
   about 2.0 wt % to about 6.0 wt % an oil-absorbing component,
   wherein the composition exhibits a maximum incremental reactivity (MIR) value of less than 0.72, and
   wherein each wt % is based on the total weight of the respective dry shampoo composition.
Statement 44. A dry shampoo composition comprising by weight percent of the total composition: about 30.0 wt % to about 90.0 wt % of a first propellant;
   about 30.0 wt % to about 90.0 wt % of a second propellant;
   about 5.0 wt % to about 40.0 wt % of at least one alcohol;
   about 1.0 wt % to about 20.0 wt % of at least one starch;
   up to about 5.0 wt % citric acid;
   up to about 5.0 wt % lactic acid;
   up to about 5.0 wt % hectorite clay;
   up to about 5.0 wt % of at least one fragrance; and
   up to about 5.0 wt % of at least one chelating agent that includes from about 35.0 wt % to about 63.9 wt % sodium polyitaconate, about 35.0 wt % to about 61.0 wt % sodium citrate and about 0.10 wt % to about 5.0 wt % Sapindus mukorossi fruit extract,
   wherein the composition exhibits a maximum incremental reactivity (MIR) value of less than 0.74.
Statement 45. The dry shampoo composition of statement 44, wherein the at least one alcohol is isopropyl alcohol present in an amount of from about 10.0% to about 12.0% by weight.
Statement 46. The dry shampoo composition of statement 44, wherein the first propellant is butane and the second propellant is propane, each present in an amount of from about 35.0% to about 45.0% by weight.
Statement 47. A dry shampoo composition comprising by weight percent of the total composition:
about 30.0% to about 90.0 wt % of a first propellant;
about 30.0% to about 90.0 wt % of a second propellant;
about 5.0% to about 40.0 wt % of an alcohol;
about 1.0% to about 20.0 wt % of a starch;
up to about 5.0 wt % of isopropyl isostearate;
up to about 5.0 wt % of sea buckthorn seed oil;
up to about 5.0 wt % of at least one odor neutralizer,
up to about 5.0 wt % hectorite clay; and
up to about 5.0 wt % of at least one fragrance,
wherein the composition exhibits a maximum incremental reactivity (MIR) value of less than 0.74.
Statement 48. The dry shampoo composition of statement 47, wherein the at least one alcohol is isopropyl alcohol present in an amount of from about 10.0 wt % to about 12.0 wt %
Statement 49. A method of refreshing or cleansing hair without water, the method including:
providing a dry shampoo composition; and
introducing or dispensing the dry shampoo composition to the scalp or hair of a subject in need thereof to reduce oiliness and refresh the hair without rinsing, wherein the dry shampoo composition includes:
   about 30.0% to about 90.0 wt % of a first propellant;
   about 30.0% to about 90.0 wt % of a second propellant;
   about 5.0% to about 40.0 wt % of at least one alcohol;
   about 1.0% to about 20.0 wt % of at least one starch;
   up to about 5.0 wt % of a citric acid;
   up to about 5.0 wt % of lactic acid;
   up to about 5.0 wt % hectorite clay;
   up to about 5.0 wt % of at least one fragrance; and
   up to about 5.0 wt % of at least one chelating agent that includes from about 35.0 wt % to about 63.9 wt % sodium polyitaconate, about 35.0 wt % to about 61.0 wt % sodium citrate and about 0.10 wt % to about 5.0 wt % Sapindus mukorossi fruit extract,
   wherein the composition exhibits a maximum incremental reactivity (MIR) value of less than 0.74, or
   about 30.0% to about 90.0 wt % of a first propellant;
   about 30.0% to about 90.0 wt % of a second propellant;
   about 5.0% to about 40.0 wt % of an alcohol;
   about 1.0% to about 20.0 wt % of a starch;
   up to about 5.0 wt % of isopropyl isostearate;
   up to about 5.0 wt % of sea buckthorn seed oil;
   up to about 5.0 wt % of at least one odor neutralizer,
   up to about 5.0 wt % hectorite clay; and
   up to about 5.0 wt % of at least one fragrance;
wherein the composition exhibits a maximum incremental reactivity (MIR) value of less than 0.74, and
wherein each wt % is based on the total weight of the respective dry shampoo composition.

What is claimed is:

1. A dry shampoo composition comprising, by weight percent of the total composition:
about 70.0 wt % to about 85.0 wt % dimethyl ether;
about 5.0 wt % to about 15.0 wt % isopropyl alcohol;
about 8.0 wt % to about 12.0 wt % rice starch;
about 0.1 wt % to about 1.0 wt % silica;
about 0.05 wt % to about 0.5 wt % isopropyl palmitate; and
optionally, about 0.05 wt % to about 1.0 wt % fragrance,
wherein the composition is substantially free of talc and water, and
wherein the composition exhibits a maximum incremental reactivity (MIR) value of less than 0.72.

2. The dry shampoo composition of claim 1, wherein the dimethyl ether is present in an amount of about 78.0 wt % to about 82.0 wt %.

3. The dry shampoo composition of claim 1, further comprising cetrimonium chloride in an amount of from about 9.0 wt % to about 11.0 wt %.

4. The dry shampoo composition of claim 1, wherein the isopropyl alcohol is present in an amount of about 9.0 wt % to about 10.0 wt %.

5. The dry shampoo composition of claim 1, wherein the silica comprises fumed silica in an amount of about 0.2 wt % to about 0.3 wt %.

6. The dry shampoo composition of claim 1, wherein the silica comprises a spherical morphology and an oil absorption capacity of at least 200 mL/100 g.

7. The dry shampoo composition of claim 1, wherein the composition is dispensed via a spraying device comprising a vapor tap orifice having a diameter of about 0.010 inches to about 0.040 inches and a spray rate of about 0.75 g/s to about 1.2 g/s.

8. A dry shampoo composition comprising, by weight percent of the total composition:
about 50.0 wt % to about 70.0 wt % dimethyl ether;
about 15.0 wt % to about 30.0 wt % water;
about 5.0 wt % to about 15.0 wt % ethanol;
about 0.1 wt % to about 2.0 wt % of a preservative comprising phenoxyethanol and ethylhexylglycerin;
about 0.005 wt % to about 0.1 wt % a polymeric film-former comprising PVM/MA decadiene crosspolymer;
about 0.001 wt % to about 0.1 wt % a pH adjusting agent comprising sodium hydroxide;
about 0.1 wt % to about 1.0 wt % glycerin;
about 0.5 wt % to about 2.0 wt % propanediol;
about 0.05 wt % to about 0.5 wt % tetrasodium glutamate diacetate;
about 0.5 wt % to about 2.0 wt % a hydrophobic emollient comprising C13-15 alkane and heptyl undecylenate;
about 0.1 wt % to about 1.0 wt % terpinolene; and
about 2.0 wt % to about 6.0 wt % an oil-absorbing component,
wherein the composition exhibits a maximum incremental reactivity (MIR) value of less than 0.72.

9. The dry shampoo composition of claim 8, wherein the oil-absorbing component comprises silica including: (a) a median particle size of less than 10 microns; and (b) a spherical morphology and oil absorption capacity of at least 200 mL/100 g;

10. The dry shampoo composition of claim 8, wherein the composition is dispensed via a spraying device comprising a vapor tap orifice having a diameter of about 0.010 inches to about 0.040 inches and at a spray rate of about 0.75 g/s to about 1.2 g/s.

11. The dry shampoo composition of claim 8, wherein the composition comprises from about 20.0 wt % to about 25.0 wt % water as a solvent.

12. The dry shampoo composition of claim 8, wherein the composition includes from about 0.30 wt % to about 0.50 wt % of a preservative selected from the group consisting of phenoxyethanol and ethylhexylglycerin.

13. The dry shampoo composition of claim 9, wherein the oil-absorbing component comprises from about 3.0 wt % to about 5.0 wt % spherical silica.

14. A dry shampoo composition comprising, by weight percent of the total composition:
about 30.0 wt % to about 90.0 wt % of a first propellant;
about 30.0 wt % to about 90.0 wt % of a second propellant;
about 5.0 wt % to about 40.0 wt % of at least one alcohol;
about 1.0 wt % to about 20.0 wt % of at least one starch;
up to about 5.0 wt % of a citric acid;
up to about 5.0 wt % of lactic acid;
up to about 5.0 wt % hectorite clay;
up to about 5.0 wt % of at least one fragrance; and
up to about 5.0 wt % of at least one chelating agent comprising: from about 35.0 wt % to about 63.9 wt % sodium polyitaconate, from about 35.0 wt % to about 61.0 wt % sodium citrate and from about 0.10 wt % to about 5.0 wt % Sapindus mukorossi fruit extract,
wherein the composition exhibits a maximum incremental reactivity (MIR) value of less than 0.74.

15. The dry shampoo composition of claim 14, wherein the at least one alcohol is isopropyl alcohol present in an amount of from about 10.0 wt % to about 12.0 wt %.

16. The dry shampoo composition of claim 14, wherein the first propellant is butane and the second propellant is propane, each present in an amount of from about 35.0 wt % to about 45.0 wt %.

17. A dry shampoo composition comprising by weight percent of the total composition:
about 30.0 wt % to about 90.0 wt % of a first propellant;
about 30.0 wt % to about 90.0 wt % of a second propellant;
about 5.0 wt % to about 40.0 wt % of an alcohol;
about 1.0 wt % to about 20.0 wt % of a starch;
up to about 5.0 wt % of isopropyl isostearate;
up to about 5.0 wt % of sea buckthorn seed oil;
up to about 5.0 wt % of at least one odor neutralizer,
up to about 5.0 wt % hectorite clay; and
up to about 5.0 wt % of at least one fragrance,
wherein the composition exhibits a maximum incremental reactivity (MIR) value of less than 0.74.

18. The dry shampoo composition of claim 17, wherein the at least one alcohol is isopropyl alcohol present in an amount of from about 10.0 wt % to about 12.0 wt %.

19. A method of refreshing or cleansing hair without water, the method comprising:
providing a dry shampoo composition; and
dispensing the dry shampoo composition to the scalp or hair of a subject in need thereof to reduce oiliness and refresh the hair without rinsing,
wherein the dry shampoo composition comprises:
about 70.0 wt % to about 85.0 wt % dimethyl ether;
about 5.0 wt % to about 15.0 wt % isopropyl alcohol;
about 8.0 wt % to about 12.0 wt % rice starch;
about 0.1 wt % to about 1.0 wt % silica;
about 0.05 wt % to about 0.5 wt % isopropyl palmitate; and
optionally, about 0.05 wt % to about 1.0 wt % fragrance,
wherein the composition is substantially free of talc and water, and
wherein the composition exhibits a maximum incremental reactivity (MIR) value of less than 0.72; or
wherein the dry shampoo composition comprises:
about 50.0 wt % to about 70.0 wt % dimethyl ether;
about 15.0 wt % to about 30.0 wt % water;
about 5.0 wt % to about 15.0 wt % ethanol;
about 0.1 wt % to about 2.0 wt % a preservative comprising phenoxyethanol and ethylhexylglycerin;
about 0.005 wt % to about 0.1 wt % a polymeric film-former comprising PVM/MA decadiene crosspolymer;
about 0.001 wt % to about 0.1 wt % a pH adjusting agent comprising sodium hydroxide;
about 0.1 wt % to about 1.0 wt % glycerin;
about 0.5 wt % to about 2.0 wt % propanediol;
about 0.05 wt % to about 0.5 wt % tetrasodium glutamate diacetate;
about 0.5 wt % to about 2.0 wt % a hydrophobic emollient comprising C13-15 alkane and heptyl undecylenate;
about 0.1 wt % to about 1.0 wt % terpinolene; and
about 2.0 wt % to about 6.0 wt % an oil-absorbing component,
wherein the composition exhibits a maximum incremental reactivity (MIR) value of less than 0.72, or
about 30.0 wt % to about 90.0 wt % of a first propellant;
about 30.0 wt % to about 90.0 wt % of a second propellant;
about 5.0 wt % to about 40.0 wt % of at least one alcohol;
about 1.0 wt % to about 20.0 wt % of at least one starch;
up to about 5.0 wt % of a citric acid;
up to about 5.0 wt % of lactic acid;
up to about 5.0 wt % hectorite clay;
up to about 5.0 wt % of at least one fragrance; and
up to about 5.0 wt % of at least one chelating agent that includes from about 35.0 wt % to about 63.9 wt % sodium polyitaconate, about 35.0 wt % to about 61.0 wt % sodium citrate and about 0.10 wt % to about 5.0 wt % Sapindus mukorossi fruit extract,
wherein the composition exhibits a maximum incremental reactivity (MIR) value of less than 0.74, or
about 30.0 wt % to about 90.0 wt % of a first propellant;
about 30.0 wt % to about 90.0 wt % of a second propellant;
about 5.0 wt % to about 40.0 wt % of an alcohol;
about 1.0 wt % to about 20.0 wt % of a starch;
up to about 5.0 wt % of isopropyl isostearate;
up to about 5.0 wt % of sea buckthorn seed oil;
up to about 5.0 wt % of at least one odor neutralizer,
up to about 5.0 wt % hectorite clay; and
up to about 5.0 wt % of at least one fragrance;
wherein the composition exhibits a maximum incremental reactivity (MIR) value of less than 0.74, and
wherein each wt % is based on the total weight of the respective dry shampoo composition.

20. The method of claim 19, wherein dispensing the dry shampoo composition comprises spraying the composition from a pressurized container through a spraying device, the spraying device comprising a vapor tap orifice having a diameter of about 0.010 inches to about 0.040 inches, and wherein the composition is dispensed at a rate of about 0.75 g/s to about 1.2 g/s.
